(19) United States
(12) Patent Application Publication
(10) Pub. No.: US 2008/0269639 A1
(43) Pub. Date: Oct. 30, 2008

(54) Device for obtaining bodily fluids for analysis

(76) Inventors: Stephan Korner, Cham (CH); Robert Partel, Baar/ZG (CH); Irio Giuseppe Calasso, Arth (CH); Desiree Decker, Egolzwil (CH)

Correspondence Address: ROCHE DIAGNOSTICS OPERATIONS INC., 9115 Hague Road, Indianapolis, IN, 46250-0457, US

(21) Appl. No.: 12/048,798

(22) Filed: Mar. 14, 2008

Related U.S. Application Data
(63) Continuation of application No. PCT/EP2006/008952, filed on Sep. 14, 2006.

(30) Foreign Application Priority Data
Sep. 15, 2005 (EP) 05020062.5

(51) Int. Cl. A61B 5/151

(52) U.S. Cl. 600/583

(57) ABSTRACT

A lancing device for piercing skin to obtain a bodily fluid, such as blood, for testing has an electrically activated actuator, such as a shape memory alloy or electro-active polymer, that changes shape upon the application of electrical energy to advance and withdraw the lancing element. The lancing device operates when the actuator changes shape to advance a lancing element to pierce the skin when the actuator changes shape, delaying the lancing element tip near the advanced position to allow fluid to flow into the lancing tip, withdrawing the lancing element to remove the lancing element form the skin when the actuator again changes shape.

### REFERENCE

This application is a continuation of PCT/EP2006/008952 filed Sep. 14, 2006 which is based on and claims priority to European Patent Application No. EP 05020062.5 filed Sept. 15, 2005, which are hereby incorporated by reference in their enthety.

### FIELD

The disclosure relates to a device for obtaining body fluid for analytical purposes comprising a lancing element that can be inserted into a body part and a drive to advance and withdraw the lancing element.

### BACKGROUND

Such withdrawal systems for small amounts of body fluids are used especially by diabetics for blood sugar self-monitoring which is carried out several times daily as part of an insulin treatment. In order to obtain capillary blood it is necessary to generate a skirt opening by a puncture while aiming to substantially reduce the puncture pain and scar formation and at the same time to ensure a hygienic procedure. In order to also enable laymen to carry out the necessary steps in a simple and rapid manner, it is desirable to achieve a substantially automated measuring process in a compact handheld device. The conventional lancing devices employ spring-driven lancing drives which are characterized by a rapid withdrawal rate of the stored energy. However, if the lancing movement should also encompass the collection of blood in so-called integrated systems, a complex movement control is necessary in order to ensure a high degree of process reliability especially when small amounts are withdrawn.

United States Patent Publication US 2004/0098009A discloses a lancing drive composed of two separate units having a mechanical (spring-driven) propulsion unit and an electrical return unit. In general the use of what is referred to there as a “nanomuscle” i.e. a shape memory alloy (SMA), is proposed for the latter, but details of specific embodiments are not given. In the quoted example the actuator is neither used for a rapid puncture movement nor for a combined forward and backward movement of a lancing element.

International Patent Application WO 02/068820 describes SMA actuators with an improved temperature control and especially concerns a shortening of the cooling times which are achieved by the special embodiment of an SMA wire in conjunction with plates as a heat sink. However, the document does not relate to the integration of an SMA actuator in lancing aids and consequently it also does not deal with the technical problems that result therefrom. The described cooling times would also not be acceptable for use in a lancing aid and a return movement would not immediately follow the lancing movement so that the lancing member would remain painfully in the skin.

### SUMMARY

The disclosure's teachings reduce the disadvantages that occur in the prior art and to enable an improved collection of body fluid using simple means.

The disclosure's teachings are based on the idea of providing a highly dynamic drive having a high energy density for a controlled advance and withdraw movement, which also may be referred to as a backward-and-forward movement. In particular a high speed should be reached when the skin is penetrated which should be rapidly and immediately converted into a return movement. Accordingly it is proposed according to the teachings that the drive has at least one actuator wire based on shape memory alloys (SMA) to control the lancing movement by means of a change in the wire length. Such SMA actuators are characterized in that they can be miniaturized and directly generate linear movements. Whereas with standard SMA actuators packed in a stack-like manner it is not possible to achieve a forward and subsequent backward movement of a lancing unit in a practical manner, it surprisingly turned out that the energy withdrawal rates and movement amplitudes of SMA wire actuators are sufficient to achieve a highly dynamic movement for a lancing and collection function. A linear lancing movement can be generated by a simple thermal influence, the small amount of heat that is required to heat up the thin wire material allows a rapid sequence of movements.

One embodiment provides that the actuator wire forms a propulsion means for the forward movement of the lancing element by heat-activated contraction. In this connection short time periods and adequate lancing depths can be achieved by just the wire design.

Embodiments can have a drive has a movement converter arranged between the actuator wire and the lancing element to convert a change in the length of the actuator wire into the lancing movement. This allows the execution of the complex movement profiles that are advantageous for a combined lancing and collection function.

The drive comprises a heating unit to heat up the actuator wire to a heating temperature which results in a contraction of the shape memory alloy. This can be achieved in a simple manner by connecting the actuator wire to a current source which generates a current pulse and in particular to a capacitor via a trigger switch. However, in principle other heating methods are conceivable for example by means of hot air, heat radiation and heat conduction, and the like.

In order to adjust the lancing depth and/or collection depth of the lancing element, it can be advantageous to be able to heat the actuator wire over a variable length preferably by means of movable electrical contact points. It can also be advantageous when, in order to adjust the lancing depth and/or collection depth of the lancing element, the actuator wire is subjected to a partial contraction by specifically heating it to a structural transformation temperature.

A type of hybrid drive can be achieved by a return means that can be pre-tensioned during the forward movement of the lancing element and in particular a return spring for the return movement of the lancing element. In general this allows the wire contraction to be converted into a forward movement and a return movement of the lancing element immediately thereafter.

Some embodiments provide that the actuator wire brakes and/or damps the return movement of the lancing element. This can improve blood collection due to the slow retraction of the lancing element.

Some embodiments have two alternately contractable actuator wires for the alternating control of the lancing movements in successive cycles.

A wire material is basically not mandatory for such an embodiment so that one aspect of the teachings are that the drive has two alternately contractable actuators based on shape memory alloys for alternately controlling the lancing movement in successive cycles.

Some embodiments have an actuator wire under contraction that drives the forward movement of the lancing element and the other actuator wire under expansion brakes and/or damps the return movement.

For a braking or damping action it is possible that the actuator wire acting as the braking means can be specifically shortened by preheating to a structural transformation temperature of the shape memory alloy and can be correspondingly elongated when it is cooled.

In some embodiments the actuator wire which brakes or damps the return movement is heated in sections.

It is also conceivable that the actuator wire acts as a braking means and/or damping means by tension-induced phase transition of the shape memory alloy.

In order to also achieve high dynamics during the return movement, some embodiments have a knee lever mechanism that can be stretched in the axis of the lancing movement or a correspondingly stretchable leaf spring. In this case a movement cycle can be achieved by means of the fact that the knee lever mechanism or the leaf spring can be brought into a stretched position by the actuator wire and into a bent position by a return element that is pre-tensioned in the stretched position.

In order to adjust the lancing depth, the bearing position of a pivot bearing of the knee lever mechanism facing away from the lancing element or of the leaf spring can be adjusted in the lancing axis.

The return movement of the lancing element can also be slowed down by a damping element in particular in the form of a piston-cylinder unit in some embodiments.

For a rapid heating it is advantageous when the diameter of the actuator wire is less than 1 mm, such as less than 0.5 mm.

In order to minimize the influence of a fluctuating ambient temperature, the shape memory alloy, winch is in particular a nickel-titanium alloy, can have a transformation temperature of more than 100° C.

In order to increase the effective wire length in the given structural space, the actuator wire can consists of several wire sections guided side-by-side over deflection means.

In order to increase the drive force, the actuator wire can consists of several individual wires running parallel to one another.

The forward movement of the lancing element for generating a skin incision can be at least an order of magnitude more rapid than the return movement for collecting body fluid.

Some embodiments can have a withdrawal device for body fluid in which the drive has an actuator formed by electro-active polymers (EAP). This also allows a highly dynamic movement sequence with a suitable lancing and collection profile in a compact construction without motor means.

The EAP actuator can have an elastomer element located between two electrodes to which a high voltage can be applied, wherein a deformation of the elastomer element caused by electrostatic attraction of the electrodes can be converted into the lancing movement.

### BRIEF DESCRIPTION OF THE DRAWINGS

The teachings of the disclosure are further elucidated in the following on the basis of the embodiment examples shown schematically in the drawing.

FIG. 1 shows a shape memory alloy (SMA) driven blood withdrawal device in a perspective view;

FIG. 2 shows different positions in the lancing movement in a side view of the embodiment according to FIG. 1;

FIG. 3 shows a time diagram of the forward and backward lancing movement;

FIG. 4 shows a circuit diagram of an electric heating unit for activating a drive for the lancing movement;

FIG. 5 shows a diagram of an SMA hysteresis cycle;

FIG. 6 shows a typical stress/elongation diagram for SMA materials;

FIG. 7 shows an electro-active polymer (EAP) driven lancing/collecting device in a schematically greatly simplified illustration; and,

FIGS. 8-10 show further embodiment examples of SMA lancing devices in a schematic representation.

### DETAILED DESCRIPTION

The lancing and collecting devices shown in the figures for collecting small amounts of blood for blood sugar tests comprise a lancing element 10 which can be inserted into a body part (for example a finger tip) that is not shown, a drive 12 for an advance and withdraw lancing movement also referred to as a forward and backward lancing movement of the lancing element 10 and a housing 14 for the drive and the linear guiding of the lancing element.

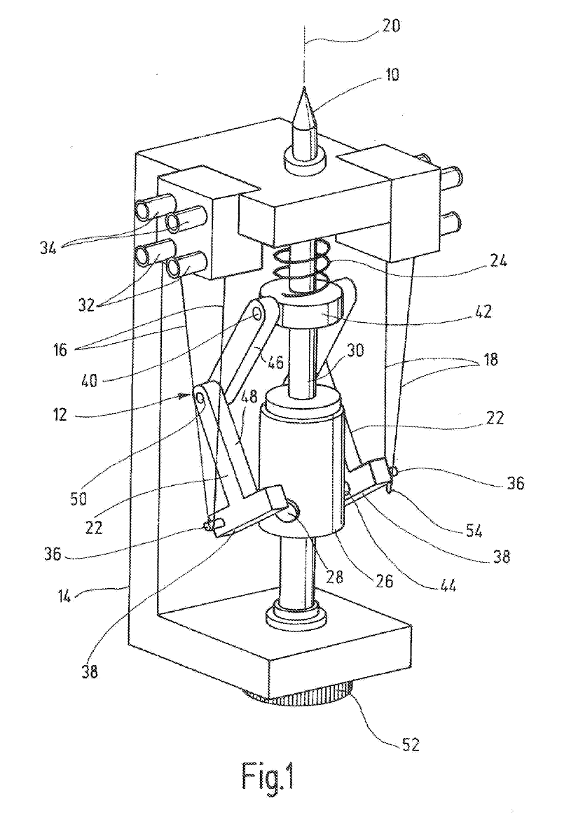

FIG. 1 shows a test setup for a drive 12 that can be actuated by means of two actuator wires 16, 18 based on shape memory alloys (SMA). When a certain transition temperature is reached, such SMA actuators change their shape and in particular the wire length is shortened which can be utilized to generate a lancing or collecting movement. For this purpose the drive 12 comprises a knee lever mechanism 22 as a motion converter which can be stretched in the axis 20 of the lancing movement. It can be brought into the stretched position in consecutive lancing cycles by the alternately contractable actuator wires 16, 18 where a reset spring 24 that is tensioned in this process ensures a return to the bent position in each cycle. Optionally a damping cylinder 26 can in this case additionally damp the return movement. The damping cylinder 26 has a valve 28 which controls the damping direction so that an undamped rapid forward movement and a damped slower return movement of the cylinder rod 30 which is guided in the cylinder is achieved.

The ends of the actuator wires 16, 18 are clamped to clamping members 32 on the housing and can by this means be electrically contacted via connecting sockets 34. A middle piece of the actuator wires 16, 18 is guided over a deflection pin 36 at one end of each arm of the T-bracket 38 of the knee lever mechanism 22 so that the two sections of wire running next to one another result in a longer wire length. In order to enable a rapid electric heating by means of the Joule effect, the wire diameter can be selected to be less than 1.0 mm such as less than 0.5 mm. In order to reduce the effect of fluctuating ambient temperatures a nickel-titanium alloy and in particular nickel-titanium-hafnium or nickel-titanium-zirconium having a transformation temperature of more than 100° C. can be used as a wire material.

The knee lever mechanism has two parallel knee lever pinions 22 which are hinge-mounted by a distal joint bearing 40 on a collar 42 of the rod 30 and are braced in a fixed position by a proximal abutment 44 during the lancing movement whereby the knee joint 50 connecting the knee levers 46, 48 swings freely. The lancing depth of the lancing element 10 can be adjusted by an adjusting device 52 which determines the position of the cylinder 26 carrying the abutment 44 in the direction of the lancing axis 20.

As already mentioned the actuator wires 16, 18 are provided for an alternating drive where the position of the wire 16 shown in FIG. 1 constitutes a propulsion means for the forward movement of the lancing element 10 by heat-activated contraction, whereas when the other wire 18 is expanded it brakes or damps the return movement of the lancing element 10. In this case the braking wire 18 can have a loop 54 which projects beyond the deflection point 36 so that the braking is not initiated until after a certain return distance.

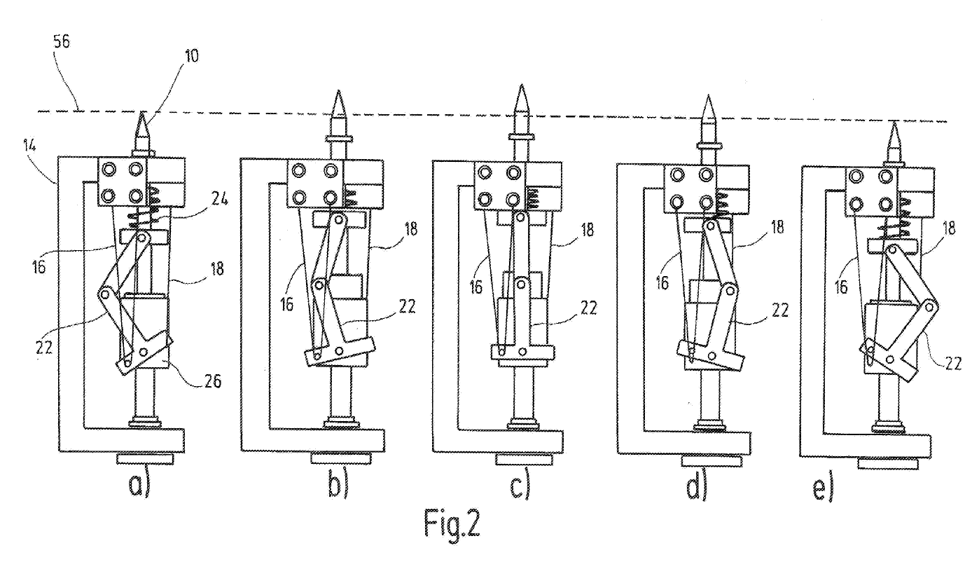

The sequence of the lancing movement is illustrated in FIG. 2 in various positions. In the starting position FIG. 2a the upper wire 16 is tensioned and the lower wire 18 is relaxed. The tip of the lancing element 10 is in the zero position 56 of the lancing stroke. Then the wire 16 is heated by a current pulse. When the transformation temperature is reached, the wire 16 contracts and the lancing element 10 moves forwards (FIG. 2b). The maximum advance position is reached in the stretched position of the knee lever mechanism 22 (FIG. 2c) in which the return spring 24 is stretched to a maximum. Then there is no significant further shortening of the wire 16 and the knee lever mechanism swings under the force of the return spring 24 into the opposing bent position FIG. 2d in which the further return movement of the lancing element 10 is delayed by the damper 26 and/or the rear actuator wire 18 in order to thus ensure a sufficient collecting time for collecting blood via a collecting channel (which is not shown as such) integrated into the lancing element. In this manner a rapid relatively pain-free puncture can be combined in one movement sequence with a collecting period that is at least an order of magnitude slower until finally the end position of FIG. 2e is reached. The wire 16 can slowly cool while the wire 18 is tensioned for the next lancing cycle without requiring a user interaction.

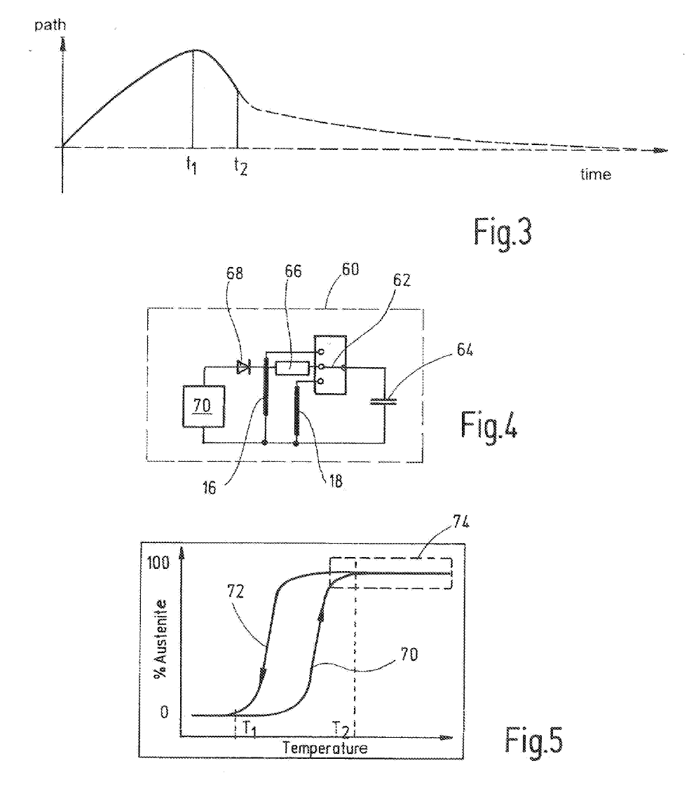

FIG. 3 again illustrates the sequence of the lancing movement in a distance-time diagram. The rapid lancing phase proceeds until a time t1, at which the maximum lancing depth is reached. For example t1 can be a few milliseconds and the lancing depth can be a few millimeters. This is followed by an initial rapid return movement until time t2 after which the body fluid is slowly collected at a suitable retracted collecting depth. This collecting phase can be in the range of seconds so that an adequate amount of fluid is collected even by capillary action alone.

In order for the knee lever mechanism 22 to swing backwards and forwards, it is necessary for the one and then the other wire 16, 18 to be heated up alternately. For this purpose the drive has an electric heating unit 60 which can be constructed according to the circuit example in FIG. 4. In the shown zero position of the change-over switch 62 the capacitor 64 in series with the resistor 66 and the diode 68 is charged from the voltage source 70 and in particular from a battery. In the upper switch position of the switch 62 the capacitor 64 can be discharged via the wire 16 which enables it to be heated in a very short period by the generated current pulse due to the low heat capacity of the thin wire. Subsequently the capacitor 64 is recharged in the zero position so that in the lower switch position the other wire 18 can now be activated.

In order to control the slow return movement during the collecting phase, the second SMA wire 16, 18 which is used in each case as a braking means can be preheated up to a certain pre-tensioning length. The return movement driven by the return spring 24 is then prematurely braked and time-delayed while the affected wire cools down. This can be achieved by a controlled heating of the shape memory alloy in the structural transformation temperature range or by partially heating only a part of the wire length.

A typical hysteresis cycle for the temperature-dependent transformation of a shape memory alloy is shown in FIG. 5 in order to further elucidate the controlled heating. During the heating-up a phase transition occurs in the metallurgical structure from the martensitic into the austenitic form according to curve 70. The reverse transition during cooling is characterized by a hysteretic behavior of the structure-temperature relationship according to curve 72. The structural change results in a tension recovery in the austenitic phase and thus a concomitant contraction into a “remembered” shape. This means that the wire length can be specifically shortened within the transformation temperature interval between T1 and T2 in order to brake the return movement of the lancing element by a corresponding expansion during cooling. In this connection the influence of the ambient temperature is problematic which can be minimized by suitable heat insulation of the wire or by selecting a material with a high conversion temperature.

In order to avoid such problems it is also conceivable to only heat segments of the respective braking or damping actuator wire. This can be achieved by appropriate electrical taps on the wire which can optionally be movably attached. The heated segment or segments are driven in an on/off actuation i.e. heated considerably above the transformation temperature interval. Hence the wire is always contracted by the same length which is then available during cooling as a defined braking distance.

In the area 74 in FIG. 5 that is framed by a dashed line which adjoins the transformation temperature interval, SMA materials exhibit a superelastic behavior under deformation. This effect is caused by stress induced martensite formation. Because the martensite component has been formed above its normal temperature, it immediately converts back into the undeformed austenite as soon as the external load is removed. This material property can also be used to damp the return movement of the lancing element 10 without large vibrations. For this purpose the wire material only has to be heated in the area 74 before the onset of the mechanical load in order that this damping then begins.

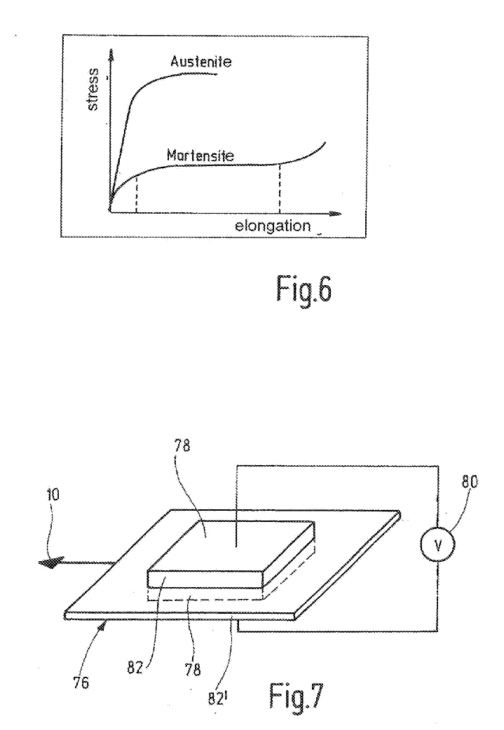

A further damping property of the SMA material occurs in the cooler martensite phase although it is less effective than the stress-induced martensite formation. According to FIG. 6 the martensite phase has a quasi-ideal damping in the damping interval marked by a dashed line which can also be utilized to slow down the return movement of the lancing element 10.

FIG. 7 illustrates the basic mode of operation of an embodiment of a lancing and collecting device based on an EAP actuator 76 formed by electroactive polymers (EAP). This has two opposing flat electrodes 78 as a thelectric EAP actuator to which high voltage can be applied via a suitable voltage source 80. An elastomer block 82 is located between the electrodes 78. The actuator principle is based on a highly dynamic deformation of this elastomer block 82 when high voltage is applied to the electrodes 78. These electrodes then attract one another due to electrostatic interaction resulting in an expansion of the incompressible elastomer material into the flat configuration 82′ with a transverse deformation. This requires elastic electrodes 78 which can accommodate the expansion in area. The transverse deformation can be utilized for a linear lancing movement of the lancing element 10. A larger stroke in the lancing axis can be achieved by a suitable geometry of the elastomer element and the coupling of the lancing element. Measurement of the capacitance by the electrodes 78 allows a feedback control of the generated movement by a closed control circuit acting on the high voltage source 80.

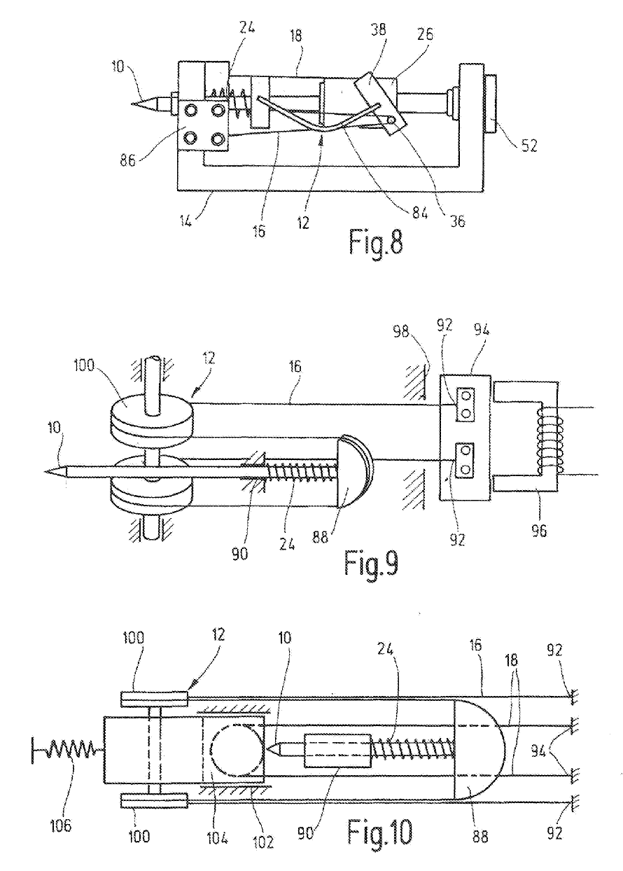

FIG. 8 shows a further embodiment example of a lancing device driven by SMA wires 16, 18 similar to the embodiment of FIG. 1 in which the same parts are labeled with the same reference numerals. In this case a leaf spring 84 is used as the knee lever mechanism 22. The ends of the spring are firmly clamped and the arm 38 is pivotally mounted about its middle axis so that a reciprocating lancing movement according to FIG. 2 is possible while the leaf spring 84 folds. In this case a knee joint axis is not necessary. This allows a further reduction in the overall size and a mechanical joint tolerance is avoided. The connecting block 86 for the SMA wires 16, 18 can advantageously be adjusted in a corresponding manner when the lancing depth is adjusted by the adjusting device 52 so that the distance from the deflection pin 36 is maintained.

FIG. 9 shows an alternative to a knee lever mechanism with only one SMA wire 16 which moves the lancing member 10 by means of a holder 88. For this purpose the holder 88 with the lancing member 10 is supported by a return spring 24 in a linear guide 90 that is fixed in the device. The two ends 92 of the wire 16 are connected to a ferromagnetic plate 94 which can be lifted by an electrode magnet 96 from a stop 98. The SMA wire 16 is multiply deflected between its ends 92 over a pivotable double deflection roller 100 and the head piece of the holder 88. When the magnet 96 is switched on, the plate 94 is attracted so that the needle 10 is advanced. When the wire 16 is subsequently shortened by a heating current pulse the needle 10 moves further forwards. At the reversal point the magnet 96 is switched off and the plate 94 strikes the stop 98 while the return spring 24 expands which results in a rapid retraction of the needle 10. The retraction speed in this case is much higher than with a mere wire cooling. In order to allow the needle 10 to reside for a period in the skin, the wire 16 can be reheated. Afterwards it is allowed to cool. This assembly has some advantages with regard to the apparatus. Thus the needle 10 can reside in the interior of a device and only be activated by the prestroke after actuating the magnet. The return spring 24 is only pre-tensioned by this means and is therefore subject to less fatigue. Furthermore, the lancing depth can be adjusted relatively simply by changing the distance between the magnet 96 and the stop 98. The overall length of the device can be shortened due to the wire deflection 100. Furthermore, when the magnet 96 is switched off the movement is damped by the SMA wire 16 which is still warm.

Two SMA wire actuators 16, 18 are provided in the embodiment according to FIG. 10 the ends of which 92 and 94 are clamped in a permanent position in the device. The longer wire 16 drives the lancing movement whereby deflection rollers 100 and a needle holder 88 corresponding to FIG. 9 are provided as deflection points. The shorter wire 18 serves to rapidly pull the needle out. again from the body part. For this purpose a deflection carriage 104 is mounted in the linear guide 102 which expands the pull-spring 106 when the wire 18 shortens and in this process the deflection rollers 100 move back in the opposite direction to lancing. As a result the needle holder 88 is moved back under the force of the return spring 24 by twice the return stroke. In order to ensure the described functionality of the drive 12 the spring constant of the pull-spring 106 must be much larger than the spring constant of the return spring 24. As a result the deflection carriage 104 only moves a little relative to the holder 88 when the wire 16 is shortened. For the lancing movement the longer wire 16 is firstly heated by a current pulse from a capacitor. As soon as this wire 16 has contracted, the second wire 18 is also heated by current induction. After a certain retraction distance the needle 10 stabilizes because the two wires 16, 18 act in an opposing manner. A line adjustment of the movement profile can simply be accomplished by adapting the pull-spring 106 and by suitable selection of the length of the wire 18.

Thus, embodiments of the device for obtaining bodily fluids for analysis are disclosed. One skilled in the art will appreciate that the teachings can be practiced with embodiments other than those disclosed. The disclosed embodiments are presented for purposes of illustration and not limitation, and the invention is only limited by the claims that follow.

What is claimed is:

1. A device for obtaining bodily fluids for analysis, comprising:
a lancing element configured for insertion into a body;
a movement converter coupled to the lancing element to control movement of the lancing element;
a drive coupled to the lancing element and the movement converter to advance and withdraw the lancing element, the drive comprising,
an electrical source coupled to the drive to selectively apply electrical energy to the drive,
at least one actuator coupled to the electrical source, the actuator changes shape upon the application of electrical energy to advance the lancing element, and the actuator changes shape upon the removal of electrical energy to withdraw the lancing element.

2. The device as in claim 1, wherein the actuator is formed from a shape memory alloy that changes shape by contracting upon the application of electrical energy causing the shape memory alloy reaches a transformational temperature.

3. The device as in claim 1, wherein the actuator is formed from an electro-active polymer that changes shape by deformation upon the application of the electrical energy causing electrostatic attraction.

4. The device as in claim 1, further comprising,
a dampener coupled to the drive to maintain the lancing element in an advanced position briefly to allow for fluid flow into a lancing tip before the lancing element moves to a withdraw position.

5. The device as in claim 1, further comprising,
a return element coupled to the drive to aid in moving the lancing element from an advance position to a withdraw position.

6. The device as in claim 1, further comprising,
a trigger switch coupled to the electrical source to provide a current pulse to the actuator causing the actuator to change shape.

7. A device for obtaining bodily fluids for analysis, comprising:
a lancing element configured for insertion into a body part;
a movement converter coupled to the lancing element to control movement of the lancing element;
a drive coupled to the lancing element and the movement converter to advance and withdraw, the drive comprising,
at least one actuator wire composed of a shape memory alloy coupled to the movement converter,
a heating element thermally coupled to the actuator wire to contract the actuator wire when a transformational temperature is reached to advance the lancing element and expand when the transformational temperature is no longer reached to withdraw the lancing element.

8. The device as in claim 7, wherein the heating element is electrical energy selectively applied by an energy source.

9. The device as in claim 7, further comprising,
a trigger switch coupled to a capacitor to provide a current pulse as the heating element to the actuator wire to cause the actuator wire to reach the transformational temperature.

10. The device as in claim 9, further comprising,
movable electrical contact points that selectively engage the actuator wire to apply electrical energy to heat only the a selected portion of the actuator wire to adjust lancing depth.

11. The device as in claim 7, further comprising,
a dampener coupled to the drive to maintain the lancing element in an advanced position briefly to allow for fluid flow into a lancing tip before the lancing element moves to a withdraw position.

12. The device as in claim 7, further comprising,
a return element coupled to the drive to aid in moving the lancing element from being advanced to being withdrawn.

13. The device as in claim 7, wherein the actuator wire is a nickel-titanium alloy with a transformational temperate greater than 100 degrees Celsius.

14. The device as in claim 7, wherein the actuator wire has a diameter of 1 mm or less.

15. The device as in claim 7, wherein the lancing element is advanced at least an order of magnitude faster than the lancing element is withdrawn.

16. A device for obtaining bodily fluids for analysis, comprising:
a lancing element configured for insertion into a body part;
a movement converter coupled to the lancing element to control movement of the lancing element; and,
a drive coupled to the lancing element and the movement converter to advance and withdraw the lancing element, the drive comprising,
means for electrical source coupled to the drive to selectively apply electrical energy to the drive,
means for actuation coupled to the electrical source, the means for actuation changing shape upon the selective application of electrical energy by the energy source to advance and withdraw the lancing element.

17. A method for operation of a lancing device to obtain bodily fluids for analysis, comprising:
positioning a lancing element in a withdrawn position;
applying electrical energy to an actuator that is coupled to the lancing element;
changing the shape of the actuator upon the application of the electrical source;
advancing the lancing element for insertion in response to the actuator changing shape;
tensioning a return spring coupled to the lancing element when the lancing element is advanced;
delaying the lancing element near the advance position to allow for fluid flow into a lancing element tip;
changing the shape of the actuator upon electrical energy no longer being applied to the actuator; and,
withdrawing the lancing element upon the actuator changing shape and through force applied by the return spring.

18. The device as in claim 17, wherein the actuator is formed from a shape memory alloy that changes shape contracting upon the application of electrical energy causeing the actuator to reach a transformational temperature.

19. The device as in claim 17, wherein the actuator is formed from an electro-active polymer that changes shape by deformation upon the application of electrical energy causing electrostatic attraction.

20. A method for operating a lancing device to obtain bodily fluids for analysis, comprising:
positioning a lancing element in a withdrawn position;
heating an upper wire by a current pulse, the upper wire being coupled to the lancing element;
contracting the upper wire when a transformational temperature is reached;
advancing the lancing element for insertion in response to the upper wire contracting;
tensioning a return spring coupled to the lancing element when the lancing element is advanced;
delaying the lancing element near the advance position to allow for fluid flow into a lancing element tip;
cooling the upper wire when by absence of the current pulse;
expanding the upper wire when the transformational temperature is no longer reached; and,
withdrawing the lancing element upon the upper wire expanding and the return spring tension.

21. The method as in claim 20, further comprising,
actuating a trigger switch coupled to a capacitor to provide the current pulse to the upper wire.
